(19) United States
(12) Patent Application Publication
(10) Pub. No.: US 2019/0267120 A1
(43) Pub. Date: Aug. 29, 2019

(54) MEDICAL DOCUMENT CREATION SUPPORT APPARATUS, METHOD, AND PROGRAM

(71) Applicant: FUJIFILM Corporation, Tokyo (JP)

(72) Inventor: Shinnosuke HIRAKAWA, Tokyo (JP)

(73) Assignee: FUJIFILM Corporation, Tokyo (JP)

(21) Appl. No.: 16/260,937

(22) Filed: Jan. 29, 2019

(30) Foreign Application Priority Data
Feb. 27, 2018 (JP) 2018-033434

(51) Int. Cl. G16H 15/00; G16H 30/20; G16H 10/60

(52) U.S. Cl. CPC G16H 15/00; G06F 17/40; G16H 10/60; G16H 30/20

(57) ABSTRACT

A document creation unit creates a medical document by inputting character information indicating the finding content for a medical image in creating a medical document for a medical image. An information acquisition unit acquires at least some of the input character information. A presentation unit presents at least one content candidate relevant to at least some of the character information with reference to medical information relevant to the medical image that includes an analysis result of the medical image. A document creation unit receives a selection of a content from the at least one content candidate and creates the medical document into which the selected content is inserted.

### CROSS REFERENCE TO RELATED APPLICATIONS

The present application claims priority under 35 U.S.C. § 119 to Japanese Patent Application No. 2018-033434 filed on Feb. 27, 2018. The above application is hereby expressly incorporated by reference, in its entirety, into the present application.

### BACKGROUND

### Field of the Invention

The present invention relates to a medical document creation support apparatus, method, and program for supporting the creation of a medical document, such as an interpretation report.

### Related Art

In recent years, advances in medical apparatuses, such as computed tomography (CT) apparatuses and magnetic resonance imaging (MRI) apparatuses, have enabled image diagnosis using high-resolution medical images with higher quality. In particular, since a diseased region can be accurately specified by image diagnosis using CT images, MRI images, and the like, appropriate treatment can be performed based on the specified result.

A medical image is analyzed by computer aided diagnosis (CAD) using a discriminator learned by deep learning or the like, regions, positions, volumes, and the like of lesions included in the medical image are extracted, and these are acquired as the analysis result. The analysis result generated by analysis processing in this manner is stored in a database so as to be associated with examination information, such as a patient name, gender, age, and a modality that has acquired the medical image, and provided for diagnosis. At this time, a technician in a radiology department or the like, who has acquired the medical image, determines a radiologist according to the medical image, and notifies the radiologist that the medical image and the result of analysis by the CAD are present. The radiologist interprets the medical image with reference to the transmitted medical image and analysis result and creates an interpretation report at his or her own interpretation terminal.

Various methods have been proposed to support the input of characters in creating a medical document, such as an interpretation report. For example, a method has been proposed in which, in a case where an operator, such as a doctor who creates a medical document, selects one or more phrases, associated phrases relevant to the selected first phrase is presented to the operator with reference to a database (refer to JP2014-002427A). In addition, a method of generating findings to be written on the interpretation report by displaying an input item name and a selection candidate group corresponding thereto based on a template defined in advance and selecting an item from the selection candidate group relevant to an organ and a lesion to be diagnosed at the time of receiving the selection input has been proposed (refer to JP2004-157815A).

However, the methods disclosed in JP2014-002427A and JP2004-157815A are to present phrases or items (hereinafter, referred to as content) relevant to character information, such as an input phrase or item, with reference to a database and a template. For this reason, the content relevant to a medical image, for which an interpretation report is to be created, is not registered in a database and a template. In particular, the analysis result of the medical image is input to the interpretation report as findings, but the content including such an analysis result of the medical image is not registered in the database and the template in the methods disclosed in JP2014-002427A and JP2004-157815A. For this reason, since the content relevant to the medical image to be interpreted is not presented even in a case where the operator inputs characters, the operator who creates an interpretation report needs to input all the characters of the content that the operator desires to input. Therefore, the methods disclosed JP2014-002427A and JP2004-157815A cannot reduce the burden on the operator.

### SUMMARY

The invention has been made in view of the above circumstances, and it is an object of the invention to reduce the burden on the operator in creating a medical document relevant to a medical image.

A medical document creation support apparatus according to the invention comprises: a document creation unit that, in creating a medical document for a medical image, creates the medical document by inputting character information indicating a finding content for the medical image; an information acquisition unit that acquires at least some of the input character information; and a presentation unit that presents at least one content candidate relevant to at least some of the character information with reference to medical information relevant to the medical image that includes an analysis result of the medical image. The document creation unit receives a selection of a content from the at least one content candidate and creates the medical document into which the selected content is inserted.

“Character information” includes not only those input by operating a keyboard, a mouse, and the like but also those input by voice.

“At least some of character information” may be only some of characters included in the character information, or may be all of the characters included in the character information.

In the medical document creation support apparatus according to the invention, the medical information may include at least one of a past interpretation report on a patient whose medical image has been acquired, an interpretation report on a similar case similar to a case included in the medical image, a medical record relevant to a patient whose medical image has been acquired, or an examination order.

In the medical document creation support apparatus according to the invention, the presentation unit may present information, which indicates a type of medical information from which each of the content candidates has been acquired, so as to be associated with the at least one content candidate.

In the medical document creation support apparatus according to the invention, the document creation unit may create the medical document including a link to the medical image.

In the medical document creation support apparatus according to the invention, the presentation unit may present a fixed sentence insertable into the medical document as the content candidate.

In the medical document creation support apparatus according to the invention, the medical document may be an interpretation report on the medical image.

A medical document creation support method according to the invention comprises: a step of creating a medical document for a medical image, in which the medical document is created by inputting character information indicating a finding content for the medical image; a step of acquiring at least some of the input character information; a step of presenting at least one content candidate relevant to at least some of the character information with reference to medical information relevant to the medical image that includes an analysis result of the medical image; and a step of receiving a selection of a content from the at least one content candidate and creating the medical document into which the selected content is inserted.

In addition, a program causing a computer to execute the medical document creation support method according to the aspect of the invention may be provided.

Another medical document creation support apparatus according to the invention comprises: a memory that stores commands to be executed by a computer; and a processor configured to execute the stored commands. The processor executes: a step of creating a medical document for a medical image, in which the medical document is created by inputting character information indicating a finding content for the medical image; a step of acquiring at least some of the input character information; a step of presenting at least one content candidate relevant to at least some of the character information with reference to medical information relevant to the medical image that includes an analysis result of the medical image; and a step of receiving a selection of a content from the at least one content candidate and creating the medical document into which the selected content is inserted.

According to the invention, at least some of the input character information is acquired, and at least one content candidate relevant to at least some of the character information is presented with reference to the medical information relevant to the medical image that includes the analysis result of the medical image. Then, the selection of the content from the at least one content candidate is received, and the medical document into which the selected content is inserted is created. For this reason, the operator can select the content that is input to the medical document from at least one content candidate relevant to the analysis result of the medical image. Therefore, it is possible to reduce the burden on the operator in creating the medical document. As a result, the operator can easily create the medical document.

### BRIEF DESCRIPTION OF THE DRAWINGS

FIG. 1 is a diagram showing the schematic configuration of a medical information system to which a medical document creation support apparatus according to an embodiment of the invention is applied.

FIG. 2 is a diagram showing the schematic configuration of the medical document creation support apparatus according to the present embodiment.

FIG. 3 is a diagram showing an interpretation report creation screen.

FIG. 4 is a diagram showing an example of presenting content candidates relevant to character information.

FIG. 5 is a diagram showing an example of characters that are selected from content candidates and inserted.

FIG. 6 is a diagram showing an example of presenting content candidates relevant to character information.

FIG. 7 is a diagram showing an example of presenting content candidates relevant to character information.

FIG. 8 is a flowchart showing a process performed in the present embodiment.

FIG. 9 is a diagram showing an example of presenting content candidates relevant to character information.

### DETAILED DESCRIPTION

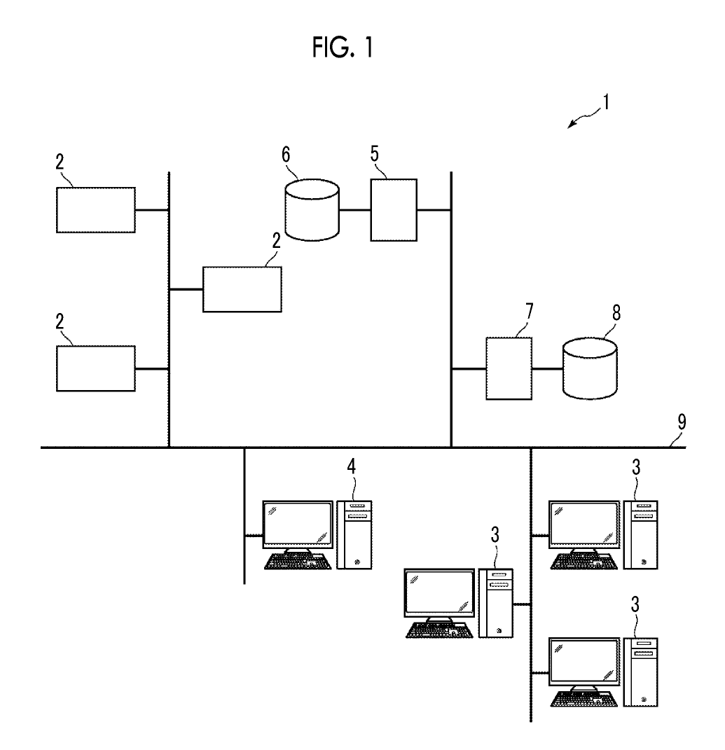

Hereinafter, an embodiment of the invention will be described with reference to the accompanying diagrams. FIG. 1 is a diagram showing the schematic configuration of a medical information system to which a medical document creation support apparatus according to an embodiment of the invention is applied. A medical information system 1 shown in FIG. 1 is a system for performing imaging of an examination target part of a subject, storage of a medical image acquired by imaging, interpretation of a medical image by a radiologist and creation of an interpretation report, and viewing of an interpretation report by a doctor in a medical department of a request source and detailed observation of a medical image to be interpreted, based on an examination order from a doctor in a medical department using a known ordering system. As shown in FIG. 1, the medical information system 1 is configured to include a plurality of modalities (imaging apparatuses) 2, a plurality of interpretation workstations (WS) 3 that are interpretation terminals, a medical department workstation (WS) 4, an image server 5, an image database 6, an interpretation report server 7, and an interpretation report database 8 that are communicably connected to each other through a wired or wireless network 9. The medical document creation support apparatus of the present embodiment is applied to the interpretation WS 3.

Each apparatus is a computer on which an application program for causing each apparatus to function as a component of the medical information system 1 is installed. The application program is recorded on a recording medium, such as a digital versatile disc (DVD) or a compact disc read only memory (CD-ROM), and distributed, and is installed onto the computer from the recording medium. Alternatively, the application program is stored in a storage device of a server computer connected to the network 9 or in a network storage so as to be accessible from the outside, and is downloaded and installed onto the computer as necessary.

The modality 2 is an apparatus that generates a medical image showing a diagnosis target part by imaging the diagnosis target part of the subject. Specifically, the modality 2 is a simple X-rays imaging apparatus, a CT apparatus, an MRI apparatus, a positron emission tomography (PET) apparatus, and the like. A medical image generated by the modality 2 is transmitted to the image server 5 and stored therein.

The interpretation WS 3 includes the medical document creation support apparatus according to the present embodiment. The configuration of the interpretation WS 3 will be described later.

The medical department WS 4 is a computer used by a doctor in a medical department to observe the details of an image, view an interpretation report, create an electronic medical record, and the like, and is configured to include a processing device, a display device such as a display, and an input device such as a keyboard and a mouse. In the medical department WS 4, each process, such as creation of a patient's medical record (electronic medical record), sending a request to view an image to the image server 5, display of an image received from the image server 5, automatic detection or highlighting of a lesion-like portion in an image, sending a request to view an interpretation report to the interpretation report server 7, and display of an interpretation report received from the interpretation report server 7, is performed by executing a software program for each process.

The image server 5 is obtained by installing a software program for providing a function of a database management system (DBMS) on a general-purpose computer. The image server 5 comprises a storage for an image database 6. This storage may be a hard disk device connected to the image server 5 by a data bus, or may be a disk device connected to a storage area network (SAN) or a network attached storage (NAS) connected to the network 9. In a case where the image server 5 receives a request to register a medical image from the modality 2, the image server 5 registers the medical image in the image database 6 in a format for a database.

Image data and accessory information of medical images acquired by the modality 2 are registered in the image database 6. The accessory information includes, for example, an image ID for identifying each medical image, a patient identification (ID) for identifying a subject, an examination ID for identifying an examination, a unique ID (UID: unique identification) allocated for each medical image, examination date and examination time at which the medical image is generated, the type of a modality used in an examination for acquiring a medical image, patient information such as patient's name, age, and gender, an examination part (imaging part), imaging information (an imaging protocol, an imaging sequence, an imaging method, imaging conditions, the use of a contrast medium, and the like), and information such as a series number or a collection number in a case where a plurality of medical images are acquired in one examination.

In a case where a viewing request from the interpretation WS 3 is received through the network 9, the image server 5 searches for a medical image registered in the image database 6 and transmits the searched medical image to the interpretation WS 3 that is a request source.

The interpretation report server 7 has a software program for providing a function of a database management system to a general-purpose computer. In a case where the interpretation report server 7 receives a request to register an interpretation report from the interpretation WS 3, the interpretation report server 7 registers the interpretation report in the interpretation report database 8 in a format for a database. In a case where a request to search for an interpretation report is received, the interpretation report is searched for from the interpretation report database 8.

In the interpretation report database 8, for example, an interpretation report is registered in which information, such as an image ID for identifying a medical image to be interpreted, a radiologist ID for identifying an image diagnostician who performed the interpretation, a lesion name, position information of a lesion, findings, and the certainty of findings, is recorded.

The network 9 is a wired or wireless local area network that connects various apparatuses in a hospital to each other. In a case where the interpretation WS 3 is installed in another hospital or clinic, the network 9 may be configured to connect local area networks of respective hospitals through the Internet or a dedicated circuit. In any case, it is preferable that the network 9 is configured to be able to realize high-speed transmission of medical images, such as an optical network.

Hereinafter, the interpretation WS 3 according to the present embodiment will be described in detail. The interpretation WS 3 is a computer used by a radiologist of a medical image to interpret the medical image and create the interpretation report, and is configured to include a processing device, a display device such as a display, and an input device such as a keyboard and a mouse. In the interpretation WS 3, each process, such as making a request to view a medical image to the image server 5, various kinds of image processing on a medical image received from image server 5, display of a medical image, analysis processing on a medical image, highlighting of a medical image based on the analysis result, creation of an interpretation report based on the analysis result, support for the creation of an interpretation report, making a request to register an interpretation report and a request to view an interpretation report to the interpretation report server 7, and display of an interpretation report received from the interpretation report server 7, is performed by executing a software program for each process. Since processes other than the process performed by the medical document creation support apparatus of the present embodiment, among these processes, are performed by a known software program, the detailed description thereof will be omitted herein. The processes other than the process performed by the medical document creation support apparatus of the present embodiment may not be performed in the interpretation WS 3, and a computer that performs the processes may be separately connected to the network 9, and requested processing on the computer may be performed according to a processing request from the interpretation WS 3.

The interpretation WS 3 includes the medical document creation support apparatus according to the present embodiment. Therefore, a medical document creation support program according to the present embodiment is installed on the interpretation WS 3. The medical document creation support program is recorded on a recording medium, such as a DVD or a CD-ROM, and distributed, and is installed onto the interpretation WS 3 from the recording medium. Alternatively, the medical document creation support program is stored in a storage device of a server computer connected to the network or in a network storage so as to be accessible from the outside, and is downloaded and installed onto the interpretation WS 3 as necessary.

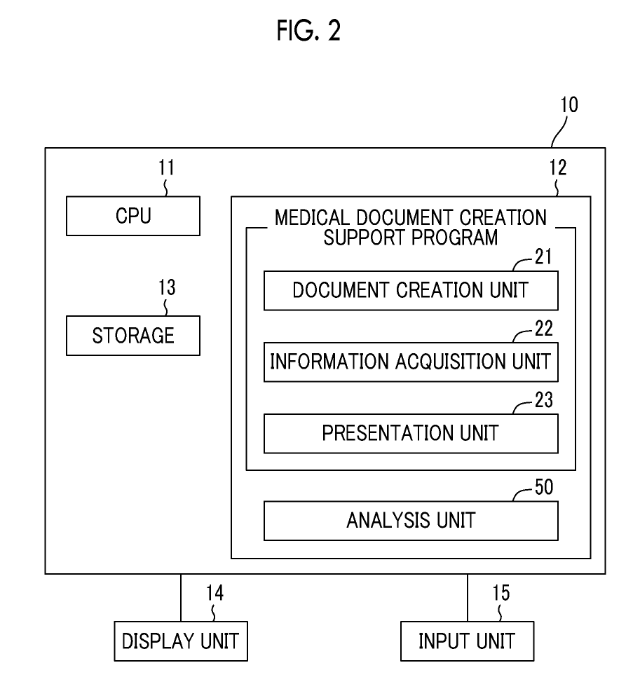

FIG. 2 is a diagram showing the schematic configuration of the medical document creation support apparatus according to the embodiment of the invention that is realized by installing the medical document creation support program. As shown in FIG. 2, a medical document creation support apparatus 10 comprises a central processing unit (CPU) 11, a memory 12, and a storage 13 as the configuration of a standard computer. A display device (hereinafter, referred to as a display unit) 14, such as a liquid crystal display, and an input device (hereinafter, referred to as an input unit) 15, such as a keyboard and a mouse, are connected to the medical document creation support apparatus 10. The input unit 15 may receive a voice input using a microphone or the like.

The storage 13 is a storage device, such as a hard disk or a solid state drive (SSD). Medical images, medical image analysis results, and various kinds of information including information necessary for processing of the medical document creation support apparatus 10, which are acquired from the image server 5 through the network 9, are stored in the storage 13.

A medical document creation support program is stored in the memory 12. As processing to be executed by the CPU 11, the medical document creation support program defines: document creation processing for creating a medical document by inputting character information indicating the finding content for a medical image in creating a medical document for the medical image; information acquisition processing for acquiring at least some of the input character information; and presentation processing for presenting at least one content candidate relevant to at least some of the character information with reference to medical information relevant to the medical image that includes an analysis result of the medical image.

The CPU 11 executes these processes according to the medical document creation support program, so that the computer functions as a document creation unit 21, an information acquisition unit 22, and a presentation unit 23. In the present embodiment, the CPU 11 executes the function of each unit according to the medical document creation support program. However, as a general-purpose processor that executes software to function as various processing units, a programmable logic device (PLD) that is a processor whose circuit configuration can be changed after manufacturing, such as a field programmable gate array (FPGA), can be used in addition to the CPU 11. Alternatively, the processing of each unit may also be executed by a dedicated electric circuit that is a processor having a circuit configuration designed exclusively to execute specific processing, such as an application specific integrated circuit (ASIC).

One processing unit may be configured by one of various processors, or may be a combination of two or more processors of the same type or different types (for example, a combination of a plurality of FPGAs or a combination of a CPU and an FPGA).

Alternatively, a plurality of processing units may be configured by one processor. As an example of configuring a plurality of processing units using one processor, first, as represented by a computer, such as a client or a server, there is a form in which one processor is configured by a combination of one or more CPUs and software and this processor functions as a plurality of processing units. Second, as represented by a system on chip (SoC) or the like, there is a form of using a processor that realizes the function of the entire system including a plurality of processing units with one integrated circuit (IC) chip. Thus, various processing units are configured by using one or more of the above-described various processors as a hardware structure.

More specifically, the hardware structure of these various processors is an electrical circuit (circuitry) in the form of a combination of circuit elements, such as semiconductor elements.

In a case where the interpretation WS 3 functions as an apparatus for performing processing other than the medical document creation support apparatus 10, a program for executing the function is stored in the memory 12. For example, in the present embodiment, an analysis program is stored in the memory 12 in order to perform analysis processing in the interpretation WS 3. Therefore, in the present embodiment, a computer that forms the interpretation WS 3 also functions as an analysis unit 50 that executes analysis processing.

The analysis unit 50 comprises a discriminator that is machine-learned to determine whether or not each pixel (voxel) in a medical image indicates a lesion and to determine the type of a lesion. In the present embodiment, the discriminator is a neural network deep-learned so as to be able to classify a plurality of types of lesions included in a medical image. The discriminator in the analysis unit 50 is learned so as to output a probability that each pixel (voxel) in a medical image is each of a plurality of lesions in a case where the medical image is input. The discriminator determines a lesion, which exceeds a predetermined threshold value and has a maximum probability, for a certain pixel, and determines that the pixel is a pixel of a lesion for which the pixel has been determined.

The discriminator may be configured to include, for example, a support vector machine (SVM), a convolutional neural network (CNN), and a recurrent neural network (RNN), in addition to the deep-learned neural network.

The analysis unit 50 specifies the type of lesion, the position of a lesion, and the size of a lesion using the determination result of the discriminator described above to generate an analysis result. For example, in a case where a medical image includes a liver, in a case where a lesion is found in the liver, the analysis unit 50 generates an analysis result including the type, position, and size of the lesion in the liver. More specifically, the analysis unit 50 generates, as an analysis result, the fact that there is a 33-mm lesion at a position S1 in the liver, the fact that there is a 20-mm lesion at a position S5 in the liver, and the like. In a case where a lesion is found, the analysis unit 50 includes information, which indicates the storage location of the analyzed medical image in the image database 6, in the analysis result. The information indicating the storage location is used to link an interpretation report as will be described later.

The document creation unit 21 creates a medical document by inputting character information indicating the finding content for a medical image in creating a medical document for the medical image. In particular, as will be described later, by receiving the selection of the content from at least one content candidate to input the medical document, a medical document into which the selected content is inserted is created. Here, the document creation unit 21 has a document creation function, such as converting input characters into Chinese characters, and creates a document based on the input from the input unit 15 by the operator. In a case where there is a voice input, the document creation unit 21 converts the voice into characters to create a document. In the present embodiment, it is assumed that an interpretation report is created as a medical document.

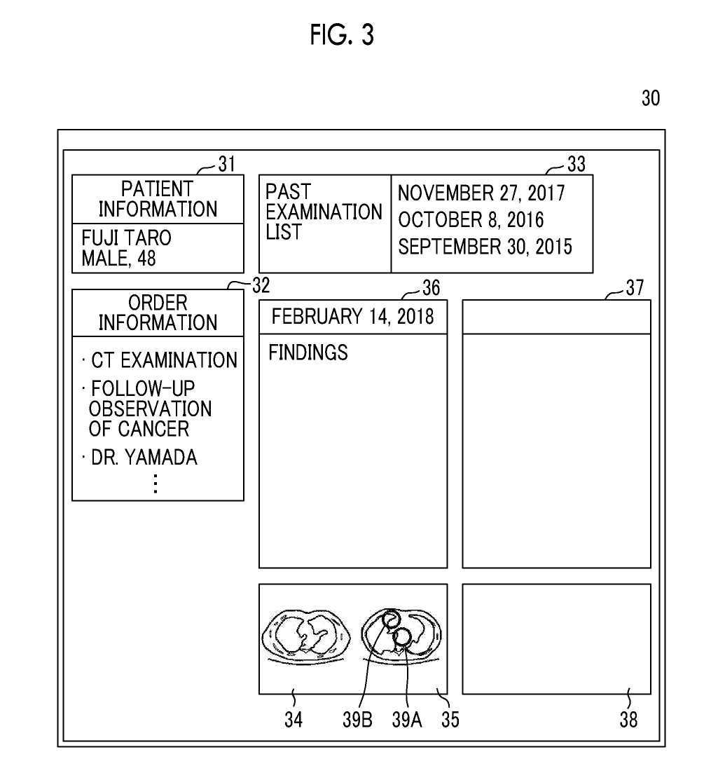

FIG. 3 is a diagram showing an interpretation report creation screen displayed on the display unit 14. As shown in FIG. 3, an interpretation report creation screen 30 includes a patient information region 31 for displaying patient information indicating the name, gender, and the like of a patient to be imaged to acquire a medical image, an order information region 32 for displaying information of an examination order for a request for an examination for acquiring a medical image, an examination list region 33 for displaying a past examination list for a patient whose medical image is acquired, medical images 34 and 35 to be interpreted, a creation region 36 for inputting a document for creating an interpretation report, a past interpretation report region 37 for displaying a past interpretation report, and a past image region 38 for displaying a past medical image for which the interpretation report displayed in the past interpretation report region 37 was created. Marks 39A and 39B for specifying the position of a lesion specified by the analysis are superimposed on the medical image 35 on the right side. The operator (radiologist) who interprets the medical images 34 and 35 inputs the sentence of the findings in the creation region 36 using the input unit 15.

The information acquisition unit 22 acquires at least some of the input character information. For example, in a case where hepatocellular carcinoma is found as a result of the interpretation of the medical images 34 and 35 by the operator, the operator inputs the characters “hepatocellular carcinoma” as findings included in the interpretation report using the input unit 15. At this time, the characters “hepatocellular carcinoma” are input. However, the information acquisition unit 22 acquires the characters “hepat”, which are the first five characters of “hepatocellular carcinoma”, as some information of the input character information.

The presentation unit 23 presents at least one content candidate, which is relevant to at least some of the character information, with reference to the medical information relevant to the medical image that includes the analysis result of the medical image. Presentation is performed by displaying a content candidate in the vicinity of a position where characters are input in the creation region 36 of the interpretation report creation screen 30.

In the present embodiment, the medical information includes analysis results of the medical images 34 and 35 performed by the analysis unit 50. As the analysis results, “there is a 33-mm lesion at the position S1 in the liver”, “there is a 20-mm lesion at the position S5 in the liver”, and the like can be used. As the medical information, it is possible to use a past interpretation report on the patient whose medical images 34 and 35 have been acquired. In this case, the presentation unit 23 sends a request for acquisition of the past interpretation report to the interpretation report server 7. The interpretation report server 7 transmits the requested interpretation report to the interpretation WS 3 with reference to the interpretation report database 8. The presentation unit 23 may send a request for acquisition of the past interpretation report to the interpretation report server 7 at a point in time at which it is determined to interpret the medical images 34 and 35 of the patient, that is, at a point in time at which a request for the medical images 34 and 35 is sent from the interpretation WS 3 to the image database 6, and store the acquired interpretation report in the storage 13.

An interpretation report on a similar case similar to the case included in the medical image may be used as the medical information. In this case, the presentation unit 23 sends a request for an interpretation report on a similar case, which is similar to the case included in the medical image, to the interpretation report server 7 with reference to the analysis results of the medical images 34 and 35. The interpretation report server 7 transmits the requested interpretation report to the interpretation WS 3 with reference to the interpretation report database 8. Alternatively, at a point in time at which the analysis results of the patient's medical images 34 and 35 are acquired, the presentation unit 23 may send a request for acquisition of a past interpretation report to the interpretation report server 7 and store the acquired interpretation report in the storage 13.

A medical record of a patient whose medical image has been acquired may be used as medical information. The medical record is stored as an electronic medical record in an electronic medical record server (not shown). The presentation unit 23 sends a request for an electronic medical record to the electronic medical record server and acquires data of the electronic medical record.

Information (order information) of an examination order for a request for an examination to acquire a medical image may be used as medical information. As the content of the examination order, order information displayed in the order information region 32 may be used. In FIG. 3, a CT examination, a follow-up observation of cancer, a name of a doctor in charge (Dr. Yamada), and the like can be used as the order information.

In addition to the analysis result of a medical image, the medical information may include at least one of a past interpretation report, an interpretation report on a similar case, an electronic medical record, or an examination order.

The presentation unit 23 presents at least one content candidate, which is relevant to some of the character information acquired by the information acquisition unit 22, in the creation region 36 with reference to the medical information. For this reason, the presentation unit 23 acquires the content candidate relevant to some of the character information from the medical information. As described above, in a case where the character information acquired by the information acquisition unit 22 is “hepat”, content candidates, such as “hepatic tumor”, “hepatocellular carcinoma”, and “hepatic fatty nodule”, are acquired from the medical information.

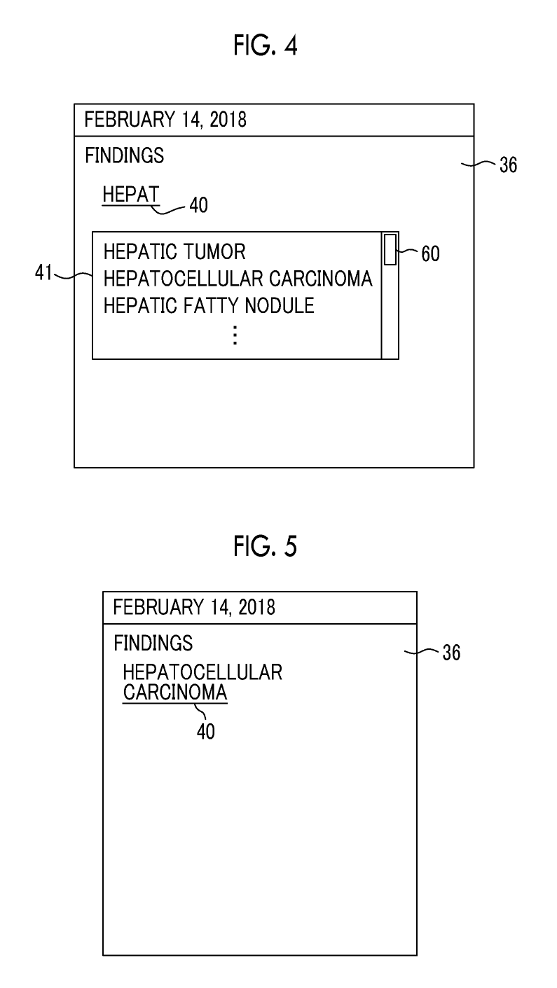

Then, the presentation unit 23 presents the acquired content candidates in the creation region 36 of the interpretation report. As described above, in a case where the character information acquired by the information acquisition unit 22 is “hepat”, the presentation unit 23 presents content candidates relevant to the character information “hepat”. FIG. 4 is a diagram showing an example of presenting content candidates relevant to the character information “hepat”. As shown in FIG. 4, the presentation unit 23 displays a candidate window 41 including content candidates below a cursor 40 in the characters “hepat” input as described above. The candidate window 41 includes content candidates, such as “hepatic tumor”, “hepatocellular carcinoma”, and “hepatic fatty nodule”. A scroll bar 60 for scrolling the displayed content candidates is displayed at the right end of the candidate window 41.

Here, in a case where the operator selects the content desired to be input from the content candidates presented in the candidate window 41, the document creation unit 21 creates a medical document into which the selected content is inserted. For example, in a case where “hepatocellular carcinoma” is selected in the candidate window 41 shown in FIG. 4, the document creation unit 21 inserts the characters “hepatocellular carcinoma” at the position of the cursor 40 in the creation region 36 as shown in FIG. 5.

On the other hand, in a case where there is no content candidate relevant to the character information acquired by the information acquisition unit 22, the presentation unit 23 presents no content candidate. In this case, the document creation unit 21 continues to create the medical document based on the input from the input unit 15 by the operator.

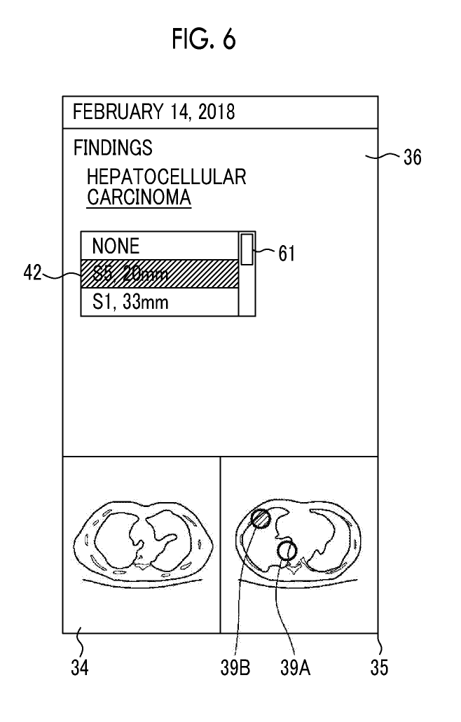

In a case where the characters “hepatocellular carcinoma” are inserted in the creation region 36, the information acquisition unit 22 acquires “hepatocellular carcinoma” as character information. The presentation unit 23 further presents content candidates relevant to “hepatocellular carcinoma” to the creation region 36 with reference to the medical information. FIG. 6 is a diagram showing an example of presenting content candidates relevant to the character information “hepatocellular carcinoma”. As shown in FIG. 6, the presentation unit 23 displays a candidate window 42 including content candidates below the cursor 40 in the characters “hepatocellular carcinoma” inserted in the creation region 36 as described above. The candidate window 42 includes content candidates of “none”, “S5, 20 mm”, and “S1, 33 mm” that are acquired by referring to the analysis result in the medical information. A scroll bar 61 for scrolling the displayed content candidates is displayed at the right end of the candidate window 42.

In this state, the operator can temporarily select the desired content in the candidate window 42 using the input unit 15. For example, in a case where the content candidate of “S5, 20 mm” is temporarily selected, the presentation unit 23 highlights the mark 39B at the position corresponding to “S5, 20 mm” in the medical image 35. In FIG. 6, temporary selection of a content candidate in the candidate window 42 is shown by hatching the temporarily selected content candidate, and highlighting of the mark 39B is shown by hatching the mark 39B. On the other hand, in a case where the content candidate “S1, 33 mm” is temporarily selected, the mark 39A at the position corresponding to “S1, 33 mm” in the medical image 35 is highlighted.

In a case where “hepatic fatty nodule” is selected in the candidate window 42 shown in FIG. 5, the document creation unit 21 inserts the characters “hepatic fatty nodule” into the creation region 36. The information acquisition unit 22 acquires the character information of “hepatic fatty nodule”, and the presentation unit 23 acquires content candidates relevant to “hepatic fatty nodule” with reference to the medical information and presents the content candidates to the creation region 36. For example, the presentation unit 23 presents content candidates, such as “no significant change” and “none”, to the creation region 36 with reference to the past interpretation report of the patient as medical information.

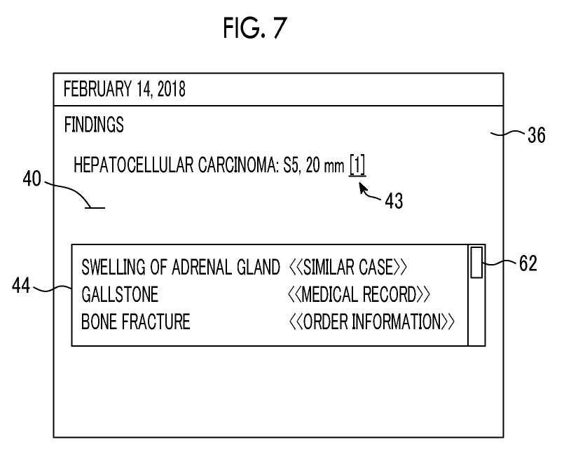

In a case where the content of “S5, 20 mm” is selected in the candidate window 42 shown in FIG. 6, the document creation unit 21 inserts “:” after the characters “hepatocellular carcinoma” in the creation region 36 and inserts the characters “S5, 20 mm” after “:” as shown in FIG. 7. The cursor 40 moves to the next line. The document creation unit 21 adds a link 43 to the medical image, in which a lesion is found at the position S5, after the inserted “S5, 20 mm”. In FIG. 7, the link 43 is shown by giving an underline to [1]. The link 43 indicates the storage location of the medical image in the image database 6, which is included in the analysis result of the medical image. In a case where the link 43 included in the interpretation report is selected after the creation of the interpretation report, the medical image of the link destination is transmitted to the interpretation WS 3, so that the medical image of the link destination is displayed on the display unit 14.

The information acquisition unit 22 acquires the character information of “hepatocellular carcinoma: S5, 20 mm”, and the presentation unit 23 acquires content candidates relevant to “hepatocellular carcinoma: S5, 20 mm” with reference to the medical information, and presents a candidate window 44 including the content candidates below the cursor 40 as shown in FIG. 7.

Here, in the candidate window 44 shown in FIG. 7, content candidates of “swelling of adrenal gland”, “gallstone” and “bone fracture” are presented. A scroll bar 62 for scrolling the displayed content candidates is displayed at the right end of the candidate window 44. After the content candidate, the type of medical information from which the content candidate has been acquired is presented. Here, it is assumed that the presentation unit 23 acquires “swelling of adrenal gland” from the interpretation report of a similar case, acquires “gallstone” from the electronic medical record of the patient, and acquires “bone fracture” from the order information. In this case, <<similar case>> that is the medical information from which the content candidate has been acquired is presented on the right side of “swelling of adrenal gland”. <<medical record>> is presented on the right side of “gallstone”, and <<order information>> is presented on the right side of “bone breakage”. Therefore, the operator can recognize from which medical information the content candidate has been acquired. In a case where the operator selects a content desired to be input from the presented content candidates, the document creation unit 21 inserts the selected content after the sentence of “hepatocellular carcinoma: S5, 20 mm”.

The document creation unit 21 can create a document regardless of the presentation of the presentation unit 23 by an input of the operator. For example, it is possible to insert the characters “seen” after the above-described “hepatocellular carcinoma: S5, 20 mm [1]” or to correct “hepatocellular carcinoma: S5, 20 mm [1]” to “hepatocellular carcinoma (S5, 20 mm [1])”. In this case, the information acquisition unit 22 further acquires at least some of the character information input by the operator, and the presentation unit 23 presents content candidates relevant to at least some of the character information with reference to the medical information.

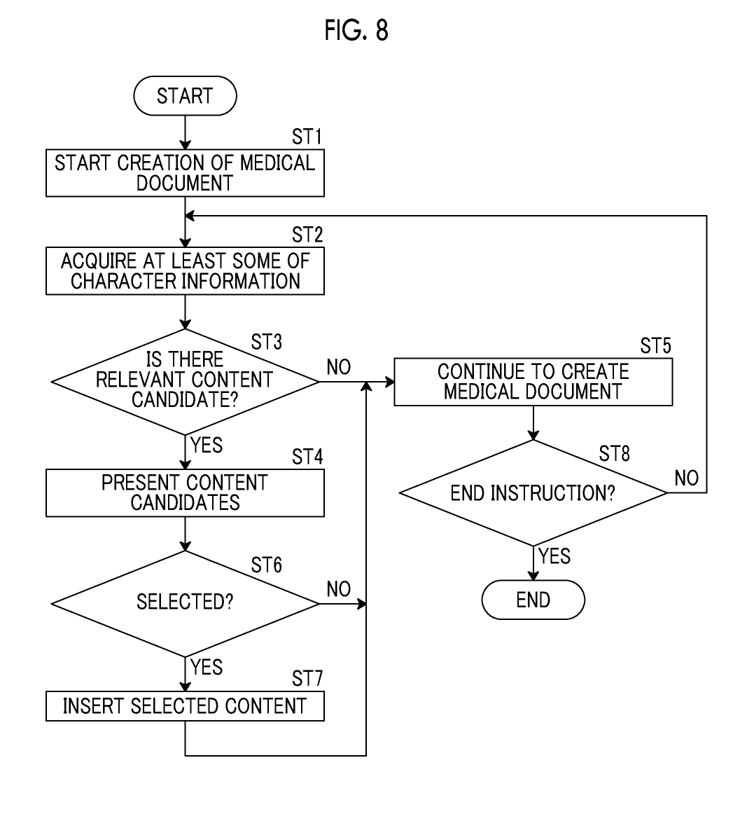

Next, a process performed in the present embodiment will be described. FIG. 8 is a flowchart showing the process performed in the present embodiment. In a case where a character input to the creation region 36 of the interpretation report is started, the document creation unit 21 starts the creation of a medical document (step ST1), and the information acquisition unit 22 acquires at least some of the input character information (step ST2). Then, the presentation unit 23 determines whether or not there is a content candidate, which is relevant to at least some of the character information, with reference to the medical information (step ST3). In a case where step ST3 is positive, the presentation unit 23 presents content candidates (step ST4). In a case where step ST3 is negative, the document creation unit 21 continues to create the medical document based on the input characters (step ST5).

The document creation unit 21 determines whether or not content has been selected from the presented content candidates (step ST6). In a case where step ST6 is positive, the document creation unit 21 inserts the selected content (step ST7), and continues to create the medical document (step ST5). On the other hand, in a case where step ST6 is negative, the document creation unit 21 continues to create the medical document (step ST5).

Subsequent to step ST5, it is determined whether or not there is an end instruction (step ST8). In a case where step ST8 is negative, the process returns to step ST2. In a case where step ST8 is positive, the process is ended.

As described above, in the present embodiment, at least some of the input character information is acquired, at least one content candidate relevant to at least some of the character information is presented with respect to the medical information relevant to the medical image that includes the analysis result of the medical image, and the medical document into which the content selected from the at least one content candidate is inserted is created. For this reason, the operator can select the content that is input to the medical document from at least one content candidate relevant to the analysis result of the medical image. Therefore, it is possible to reduce the burden on the operator who creates the medical document. As a result, the operator can easily create the medical document.

In particular, in a case where the medical document is an interpretation report, the interpretation report includes the analysis result of the medical image as the findings. In the present embodiment, content candidates are presented with reference to the medical information including the analysis result of the medical image. Therefore, according to the present embodiment, it is possible to further reduce the burden on the operator who inputs the analysis result in creating the interpretation report.

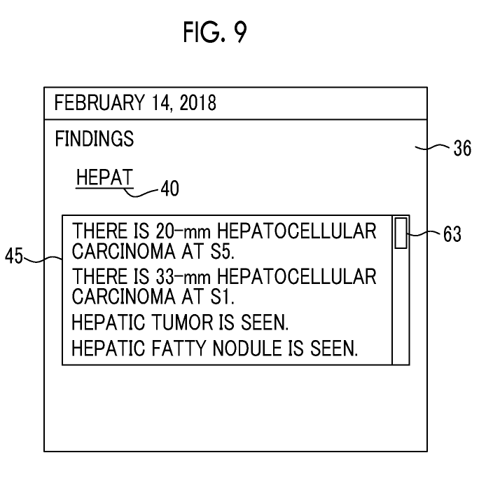

In the embodiment described above, the presentation unit 23 presents content candidates to be inserted into the medical document from the input character information, the invention is not limited thereto. The presentation unit 23 may present a fixed sentence, which can be included in the interpretation report as it is, as a content candidate relevant to the input character information. For example, in a case where the characters “hepat” are input as described above, a candidate window 45 including fixed sentences of “there is 20-mm hepatocellular carcinoma at S5”, “there is 33-mm hepatocellular carcinoma at S1”, “hepatic tumor is seen”, and “hepatic fatty nodule is seen” may be presented as content candidates below the cursor 40 as shown in FIG. 9. A scroll bar 63 for scrolling the displayed content candidates is displayed at the right end of the candidate window 45. In this case, in a case where the operator selects the desired content from the content candidates presented in the candidate window 45, the document creation unit 21 inserts the content candidate selected by the operator into the interpretation report as it is. By presenting the fixed sentence as a content candidate as described above, it is possible to further reduce the burden on the operator who creates the medical document.

In the embodiment described above, content candidates are presented, for example, by inputting the characters “hepat”. However, content candidates may be presented by inputting the first character “h” of “hepat”.

In the embodiment described above, in creating the interpretation report as a medical document, the invention is applied. However, it is needless to say that the invention can be applied even in the case of creating medical documents other than the interpretation report, such as an electronic medical record and a diagnosis report.

In the embodiment described above, the analysis unit 50 for analyzing a medical image is provided in the interpretation WS 3, but the invention is not limited thereto. In the interpretation WS 3, a result of the analysis of an external analysis apparatus may be acquired, and the acquired analysis result may be used as medical information for presenting a content candidate.

What is claimed is:

1. A medical document creation support apparatus, comprising:
a document creation unit that, in creating a medical document for a medical image, creates the medical document by inputting character information indicating a finding content for the medical image;
an information acquisition unit that acquires at least some of the input character information; and
a presentation unit that presents at least one content candidate relevant to at least some of the character information with reference to medical information relevant to the medical image that includes an analysis result of the medical image,
wherein the document creation unit receives a selection of a content from the at least one content candidate and creates the medical document into which the selected content is inserted.

2. The medical document creation support apparatus according to claim 1,
wherein the medical information includes at least one of a past interpretation report on a patient whose medical image has been acquired, an interpretation report on a similar case similar to a case included in the medical image, a medical record relevant to a patient whose medical image has been acquired, or an examination order.

3. The medical document creation support apparatus according to claim 1,
wherein the presentation unit presents information, which indicates a type of medical information from which each of the content candidates has been acquired, so as to be associated with the at least one content candidate.

4. The medical document creation support apparatus according to claim 1,
wherein the document creation unit creates the medical document including a link to the medical image.

5. The medical document creation support apparatus according to claim 1,
wherein the presentation unit presents a fixed sentence insertable into the medical document as the content candidate.

6. The medical document creation support apparatus according to claim 1,
wherein the medical document is an interpretation report on the medical image.

7. A medical document creation support method, comprising:
a step of creating a medical document for a medical image, in which the medical document is created by inputting character information indicating a finding content for the medical image;
a step of acquiring at least some of the input character information;
a step of presenting at least one content candidate relevant to at least some of the character information with reference to medical information relevant to the medical image that includes an analysis result of the medical image; and
a step of receiving a selection of a content from the at least one content candidate and creating the medical document into which the selected content is inserted.

8. A non-transitory computer-readable storage medium that stores a medical document creation support program causing a computer to execute:
a step of creating a medical document for a medical image, in which the medical document is created by inputting character information indicating a finding content for the medical image;
a step of acquiring at least some of the input character information;
a step of presenting at least one content candidate relevant to at least some of the character information with reference to medical information relevant to the medical image that includes an analysis result of the medical image; and
a step of receiving a selection of a content from the at least one content candidate and creating the medical document into which the selected content is inserted.
